(19) United States
(12) Patent Application Publication
(10) Pub. No.: US 2020/0163869 A1
(43) Pub. Date: May 28, 2020

(54) Photogenic: The Hair Care System in a Jar (Minus Cleanser)

(71) Applicant: Emery McClendon, Studio City, CA (US)

(72) Inventor: Emery McClendon, Studio City, CA (US)

(21) Appl. No.: 16/198,766

(22) Filed: Nov. 22, 2018

(51) Int. Cl. A61K 8/9789; A61Q 5/12; A61Q 5/06; A61K 8/81; A61K 8/9794; A61K 8/34; A61K 8/67; A61K 8/368; A61Q 7/00; A61Q 19/08; A61Q 5/00

(52) U.S. Cl. CPC A61K 8/9789; A61Q 5/12; A61Q 5/06; A61K 8/8147; A61K 8/9794; A61K 8/345; A61K 2800/5922; A61K 8/368; A61Q 7/00; A61Q 19/08; A61Q 5/004; A61K 2800/30; A61K 8/678

(57) ABSTRACT

This disclosure relates generally to novel system comprised to provide leave-in conditioning hair styling product that improves the style and also coats, conditions and moisturizes. Also provided the composition of the present invention is a method of controlling the frizz condition. The styling gel compositions impart excellent hair styling, conditioning, and hair set retention properties to treated hair. The favorable characteristics of the composition include no drag on comb-out and a clean feel on the hair after use.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

### Field of the Invention

The present invention relates to leave-in conditioning styling gel (Photogenic) that is applied to the hair to shape, style, moisturizes and condition the hair. The invention has excellent hair styling, combing, and style retention properties, and is easily washable from treated hair.

### Description of the Related Art

Human hair becomes soiled due to its contact with the surrounding atmosphere and, to a greater extent, from sebum secreted by the head. The build-up of the sebum causes the hair to have a dirty feel and an unattractive appearance. The soiling of the hair necessitates it being shampooed with frequent regularity.

The condition and appearance of hair can be improved by applying a composition that conditions the hair and helps maintain the hair in a predetermined configuration, or hairstyle. Hair setting and conditioning can be achieved by applying such a composition to wet hair, fixing the hair by drying, then combing to give finishing touches and provide the desired hairstyle. Similarly, after applying the composition to the hair, the wet hair can be set by using any of a variety of rollers or curlers to mechanically fix the hair in a predetermined configuration before drying. In either case, the wet hair is dried, either by ambient air drying, electric drying, or hot air, i.e., blow, drying, to set the hair.

The inherent problem encountered in hair setting is the natural tendency of hair to return to its natural shape. For example, set hair returns to its natural shape almost immediately if moistened. Likewise, high humidity conditions accelerate the tendency of hair to return to its natural shape. Therefore, intensive efforts have been directed toward providing a hair set with sufficient holding power to maintain the desired hairstyle until at least the next shampoo, and, accordingly, giving the hair set a degree of permanency.

One type of hair styling composition is a hair spray product. Hair spray products are applied to wet and/or dry hair and contain a polymer, or polymer mixture, that remains fixed on the previously styled hair and effects the hair in various ways. For example, a “mechanical” effect is exerted on each individual hair. The film-forming polymers are used to provide a flexible sheath of polymeric film on the shaped hair after drying, and, therefore, for mechanical reasons, retard the return of each individual hair to its natural shape. In addition, the polymeric film provides an overall stiffening of the hair. The hair behaves as if the individual hair strands are welded together, and the final hairstyle has better cohesion, therefore, resisting the natural forces that return the hair to its natural shape. Finally, the polymeric film protects the hair from humidity. The ability of the polymeric film to attract and absorb water preferably is minimal, such that the polymeric film retards moisture uptake by hair and retards the return of the hair to its natural state.

Another styling composition relates to wide variety of hair styling gels that are available for hair fixation, particularly for creating spikes and the like, which can add body and shine to hair without unwanted flaking or dryness. These formulations generally allow the creation of “hair looks” such as the wet or dry looks, to control frizzie perms and natural waves, to provide long lasting blow waves which simulate a non-oil wet look and which when brushed through can create a dry fluffy look.

However, to Applicant's knowledge, none of these prior art methods have been found to be completely suitable to meet these needs and are cumbersome. In view of the drawbacks associated with conventional hair styling compositions, there is an ongoing need for hair styling compositions. The overall combination of these features is nowhere disclosed in the prior art cited above which appears to be representative of the general art in this area although it is not intended to be an all-inclusive listing of pertinent prior art patents.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

According to a first embodiment the present invention is an all-in-one product. When applied, it coats the hair strands in nutrients, deep conditioning your hair 24/7. Even though Photogenic doesn't dry hard, it has great hold and hair remains nourished and shiny after Photogenic dries. Being chemically free in nature this product does not chemically alter your hairs.

The hair styling gels impart good hair set retention and a soft, natural feel to treated hair, and provide superior hairstyle retention at high relative humidity. The hair styling gels also detangle hair and condition the hair. Such results are unexpected because traditional hair setting resins are hydrophobic. In contrast, the invention ingredients provide a soft, natural feel to the hair, and the hair is not tacky.

Another embodiment of the invention is, a formulation that styles, conditions and grow hairs.

Another embodiment of the invention is, moisturizing leave-in conditioners, oils, and styling gels to products claiming to provide curl definition, reduce shedding, and repair damage.

Another embodiment of the invention is, further directed to a method of treating hair and to the use of the leave-in hair styling product for improving the volume of a hair-do.

Embodiments of the invention relates to a clear hair styling gel composition of the invention provides effective hair fixture properties with enhanced clean feel and shine without drag on comb-out, oiliness or tackiness.

The clear hair styling gel composition of the invention provides effective hair fixture properties with enhanced clean feel and shine, even at low copolymer gel levels, without drag on comb-out, oiliness or tackiness.

The invention works great on skin. It moisturizes extremely dry skin and can also be used for facials and to combat wrinkles.

Many objects of this invention will appear from the following description and appended claims, reference being made to the accompanying drawings forming a part of this specification wherein like reference characters designate corresponding parts in the several views.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 100 shows the result of pre-photogenic and post-photogenic.

FIG. 200 shows different applications before and after use.

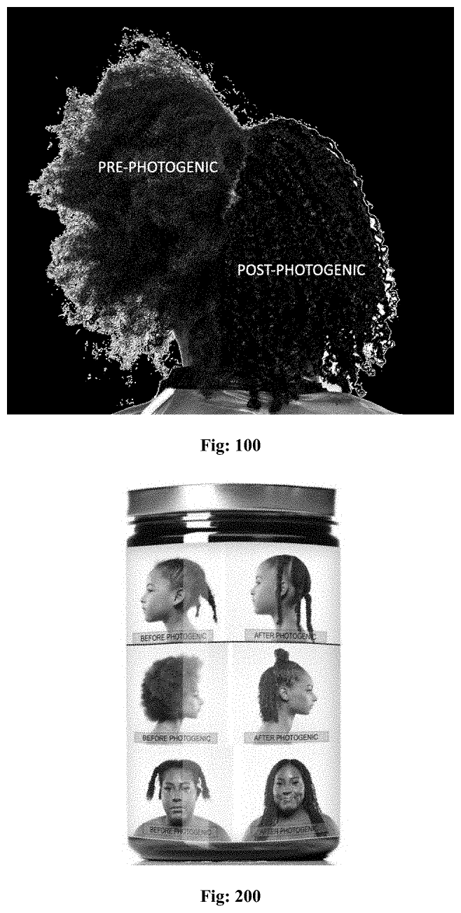

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Before explaining the invention in detail, it is to be understood that the invention is not limited in its application to the detail of application illustrated in the drawings since the invention is capable of other embodiments and of being practiced or carried out in various ways. It is also to be understood that the phraseology or terminology employed is for the purpose of description only and not of limitation.

Embodiments of the present invention relate to unique, cost effective and efficient hair treatment system of the hair gel composition defined to include constituents that are Water, Carbomer, Shea Butter, Coconut Oil, Propylene Glycol, Soy Protein, Wheat Protein, Glycerin, Mango Seed, Aloe, Avocado Oil, Kernel Oil, Neem Seed, Hibiscus Flower, Carrot Oil, Vitamin E, Benzoic Acid, Olive Oil, Fragrance & Color.

Accordingly, an advantage of the present invention is that it provides for a hair gel having an ingredient composition that also includes a fragrance, a moisturizer, coats and conditions. The moisturizing leave-in conditioners, oils, and styling gels to products claiming to provide curl definition, reduce shedding, and repair damage. The invention is a hair product that does the work of a conditioner, deep conditioner, leave in conditioner and detangler all in one simple step.

Another advantage of the present invention is that it provides for a hair gel that is made from hair grooming ingredients that are hypoallergenic and safe for the user to apply to the hair and scalp.

A further advantage of the present invention is that it provides for a hair gel that is long-lasting, durable in application and reliable in providing hair grooming and conditioning. The invention helps in reducing the emergence of gray hairs and growing longer hairs.

The hair styling compositions of the present invention are used in conventional ways to provide the hair styling benefits of the present invention. Such method generally involves application of an effective amount of the styling product to wet or dry hair. By “effective amount” is meant an amount sufficient to provide the hair style benefits desired considering the length and texture of the hair. After the hair is treated with the compositions of the present invention, the hair is styled in the usual ways of the user. Said method preferably comprises the steps of:

1. providing a leave-in hair styling product according to the invention as described above,

2. applying the composition of said leave-in hair styling product to dry hair before, and

3. leaving the composition on the hair to dry to style hair as desired.

Such method generally involves application of an effective amount of the product to dry, slightly damp, or wet hair preferably before the hair is arranged to a desired style. The composition is then dried or allowed to dry. By “effective amount” is meant an amount sufficient to provide the hair volume and style benefits desired considering the length and texture of the hair.

A few of the finest points of the invention are:

Photogenic works best on dry or very slightly damp hair (it already contains the appropriate amount of water to work correctly).

If user have at least shoulder-length hair, until your hair dries, cover your shoulders with a cape or something that won't absorb the product from the ends of your hair (a major cause of shrinkage).

If user want a more defined look or if you are not getting enough hair elongation, add more Photogenic, focusing on the roots and the ends of the hair (coat your ends a bit more than the rest).

Add more Photogenic than you think you'll need, and you'll retain shine and definition when your hair dries. Everyone's hair is different, so the amount will vary, but you have to add enough for it to work. Remember, you are replacing all your wash day hair products, so apply Photogenic generously.

Photogenic is amazing when used for: roller sets, braiding, twist outs, revitalizing wigs+extensions, maintaining almost any hairstyle between salon visits, and conditioning your hair while in protective styles. Photogenic is also amazing at adding shine and luster to naturally straight or relaxed hair.

Photogenic replaces your leave-in styling gel. It has a slight hold to keep curls and waves defined. Do not attempt to comb through your hair once it dries if you've used Photogenic to mold your hair or lay it straight. Instead, moisturize your hair with water first. This will reactivate the Photogenic on your hair, allowing the slicked hair to soften and move.

The method can be used to treat and prevent conditions such as male pattern baldness, hair loss due to aging, or hair loss due to chemotherapy or drug exposure. The invention can be used as blowout cream to make hair silky and shiny, fight fizz and to speed up drying time

A typical formulation of the invention consist of Natural ingredients that will bring user hair to optimal health were used in the production of Photogenic. The first ingredient is water, which is a clear indication of the moisture that will be penetrated into your hair shafts. It also contains: shea butter, coconut oil, soy & wheat protein, mango seed, aloe, avocado & kernel oil, neem seed, hibiscus flower, carrot & olive oil, and vitamin e. These ingredients will soften and moisturize the hair, repair and strengthen damaged hair shafts and cuticles, stimulate growth, detangle and add shine.

The compositions of the present invention may further comprise one or more optional components known for use in hair care or personal care products, provided that the optional components are physically and chemically compatible with the essential components described herein, or do not otherwise unduly impair product stability, aesthetics or performance. Individual concentrations of such optional components may range from about 0.001% to about 10% by weight of the compositions.

In addition to the ingredients described above, a mixture of cleansing ingredients as well as other appropriate functional ingredients (preservatives, fragrance, water, etc.) have been added to formulations in such a way as to deliver the desired styling activity for the hair and scalp in a consumer-acceptable form. In a similar way, the ingredients described above have been added to a mixture of conditioning ingredients and other appropriate functional ingredients have been added to each of the conditioner formulations in such a way as to deliver the desired conditioning activity for the hair in a consumer-acceptable form.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1) The present invention is a leave-in conditioning styling gel comprising water, Carbomer, Shea Butter, Coconut Oil, Propylene Glycol, Soy Protein, Wheat Protein, Glycerin, Mango Seed, Aloe, Avocado Oil, Kernel Oil, Neem Seed, Hibiscus Flower, Carrot Oil, Vitamin E, Benzoic Acid, Olive Oil, Fragrance & Color;
a. The styling gel of claim 1, provides a method for stimulating hair follicle growth;
b. The styling gel of claim 1, wherein the hair care formulation includes a function of conditioner formulation, revitalizing conditioner formulation, and scalp treatment formulation;
c. The styling gel of claim 1, that does the work of a conditioner, deep conditioner, leave in conditioner and detangler all in one simple step;
d. The styling gel of claim 1, work by miniaturizing effect that can shrink large adult hairs, resulting in finer hairs for styling;
e. The styling gel of claim 1, that does not chemically alter your hair and is free of any harmful chemicals;
f. The styling gel of claim 1, leaving the composition on the hair without subsequent rinsing;
g. The styling gel of claim 1, works on all types of hairs whether it's a braid or general hair style;
h. The styling gel of claim 1, can be used for blowout hairstyle on straight hairs;
i. The styling gel of claim 1, can be used on natural hair—both on user beard and on head;
j. The method of claim 1, wherein the hair care formulation is the scalp treatment formulation.

2) providing a leave-in hair styling product according to any of the product claims.

3) an all-in-one product which does not need any other substance of product to be effective;
a. The all-in-one of claim 3, intends to reduce the appearance of lines and wrinkles also works great as a moisturizes on skin.

4) The invention can be used as blowout cream to make hair silky and shiny, fight frizz and to speed up drying time.

5) The invention act as a heat protectant providing an extra layer of protection against damage that can be caused by heat.
